(19) United States
(12) Patent Application Publication
(10) Pub. No.: US 2023/0088127 A1
(43) Pub. Date: Mar. 23, 2023

(54) ISOTOPES OF ALPHA KETOGLUTARATE AND RELATED COMPOUNDS AND THEIR USE IN HYPERPOLARIZED IMAGING

(71) Applicant: THE UNITED STATES OF AMERICA, AS REPRESENTED BY THE SECRETARY, DEPARTMENT OF HEALTH AND HUMAN SERVIC, Bethesda, MD (US)

(72) Inventors: Chandrasekhar Mushti, Germantown, MD (US); Deepak Sail, Potomac, MD (US); Rolf Eric Swenson, Silver Spring, MD (US); Murali Krishna Cherukuri, Potomac, MD (US); Shingo Matsumoto, Sapporo City (JP); Aparna Kesarwala, Altanta, GA (US)

(21) Appl. No.: 17/793,089

(22) PCT Filed: Jan. 15, 2021
(86) PCT No.: PCT/US2021/013658

§ 371 Date: Jul. 15, 2022

Related U.S. Application Data
(60) Provisional application No. 62/962,473, filed on Jan. 17, 2020.

(51) Int. Cl. C07B 59/00; A61K 45/06; A61K 51/04

(52) U.S. Cl. CPC C07B 59/001; A61K 45/06; A61K 51/0402

(57) ABSTRACT

A compound of the Formula I or a pharmaceutically acceptable salt thereof, wherein R₁, Ca, Cb, Cd, and n are the same as described in the specification. Disclosed is a method of diagnosing or monitoring a patient suffering from cancer, the method comprising: administering a pharmaceutical composition comprising an effective amount of an active agent, wherein the active agent is the compound of Formula I, a pharmaceutically acceptable salt of any of the foregoing thereof, or a combination thereof, together with a pharmaceutically acceptable carrier to the patient and diagnosing or monitoring the patient by hyperpolarized ¹³C-MRI. Also disclosed is a method of synthesizing 1-¹³C-5-¹²C-diacid.

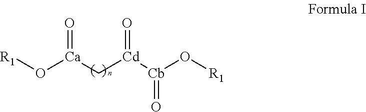

### CROSS REFERENCE TO RELATED APPLICATIONS

This application claims priority from U.S. Provisional Application No. 62/962,473, filed 17 Jan. 2020, which is hereby incorporated by reference in its entirety.

### STATEMENT OF GOVERNMENT SUPPORT

This invention was made in part with government support from the National Institutes of Health. The government has certain rights in this invention.

### BACKGROUND OF THE INVENTION

### 1. Field of the Invention

The present invention is directed to isotopically labled alpha ketoglutarate and related compounds of Formula I as well as their use as a hyperpolarized imaging, therapeutic, or diagnostic agent and methods for preparation.

### 2. Brief Description of the Related Art

Atypical metabolism of alpha-ketoglutarate (α-KG) has been linked to increased tumor cell differentiation, amplified malignant progression, as well as altered protein synthesis and catabolism. Morris J. Yashinkie J, Koche R et al. α-ketoglutarate links p53 to cell fate during tomour suppression. Nature 2019; 573(7775): 595-599; Wu N, Yang M, Gaur U et al. Alpha-ketoglutarate: Physiological Functions and Applications. Biomol Ther (Seoul). 2016; 24(1): 1-8. α-KG is also the substrate for the mutant isocitrate dehydmgenase I (IDH1) enzyme. IDH1 is a cytosolic enzyme that catalyzes the oxidation of isocitrate to alpha-ketoglutarate. Mutations in this enzyme, most commonly a heterozygous point mutation of arginine 132 to histidine (R 132H), allow for the reduction of α-KG to oncometabolite 2-hydroxyglutarate (2-HG). This gain-of-function mutation leads to a buildup of 2-HG in IDH 1 mutant cells. 2-HG can also be produced by non-canonical functions of lactate dehydrogenase (LDH) and malate dehydrogenase (MDH), but these promiscuous reactions by LDH and MDH do not typically lead to high concentrations of 2-HG in cells. As 2-HG acts as an inhibitor of α-KG-dependent dioxygenases, the high concentrations of 2-HG in IDH1 mutant cells can have multiple downstream effects. Xu W, Yang H, Liu Y et al. Oncometabolite 2-hydroxyglutarate is a competitive inhibitor of alpha-ketoglutarate-dependent dioxygenases. Cancer Cell 2011: 19: 17-30.

Roughly 80% of grade II and III glioma and secondary glioblastoma tumors contain mutations in IDH1. Similar mutations have been found in other tumor types including acute myeloid leukemia, chondrosarcoma and intrahepatic cholangiocarcinoma as well as in colorectal and pancreatic adenocarcinomas. As IDH1R 132 mutations correlate with hypermethylation, increased radiosensitivity, and a less aggressive phenotype, IDH1 status has become an important indicator for patient stratification and tumor classification.

In the absence of a gain of function mutation in IDH1, concentrations of 2-HG are typically below detectable levels by magnetic resonance spectroscopy (MRS) or magnetic resonance imaging (MRI) methods, which allows for the high concentrations of 2-HG in IDH1 mutant cells to be a unique indicator of IDH1 status in vivo. MRI is a technique that can be used to measure 2-HG production in both biopsy samples and glioma patients in vivo. While in vivo ¹H MRI in principle offers a method for measuring 2-HG levels, overlapping resonances and the general insensitivity of 1H MRI has made accurate quantitation difficult in many cases.

In 2013, Chaumeil and colleagues developed a complementary approach, measuring the real-time metabolism of α-KG to 2-HG and glutamate by injecting [1-¹³C]-α-KG into rats bearing glioblastoma xenografts. (Chaumeil, M. M. et al. Non-invasive in vivo assessment of IDH1 mutational status in glioma. Nature communications, 4, 2429, doi:10.1038/ncomms3429 (2013). When IDH1 mutant enzymes are present, [1-¹³C[-2-HG is produced and can be measured via hyperpolarized carbon-13 MRI (HP-¹³C-MRI). HP-¹³C-MRI has become an important tool in the study of real-time metabolism in vivo, as hyperpolarization allows for an over 10,000-fold enhancement of MRI signal of ¹³C labeled molecules. To perform HP-¹³C-MRI studies, a high concentrated solution of the desired metabolite is cooled by liquid helium, polarized using a super-conducting magnet, and subsequently rapidly dissolved in a pH neutralizing solution. The hyperpolarized sample can then be injected either in vitro or in vivo, and metabolism of the sample can be followed by MRI. Currently, HP-¹³C-MRI is being used to track lactate production from injected hyperpolarized [1-¹³C]-pyruvate to noninvasively diagnose cancer, image tumor location, and monitor response to therapy in patients with prostate cancer.

However, the HP-¹³C-MRI signal from the naturally-present ¹³C at the C5 position of [1-¹³C]-α-KG overlaps with the signal of [1-¹³C]-2-HG produced by IDH1 mutant enzyme, making the detection of [1-¹³C]-2-HG and therefore characterization of the IDH 1 status difficult. Therefore, there is a strong interest in developing [1-¹³C]-α-KG derivatives that can avoid signal overlap and improve the sensitivity of analysis.

### SUMMARY OF THE INVENTION

In one aspect, the present invention is directed to a compound of Formula I

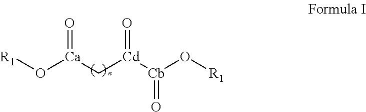

or a pharmaceutically acceptable salt thereof,

wherein in Formula I

R₁ is hydrogen, C₁-C₆alkyl, C3-C7cycloalkyl, (C3-C7cycloalkyl)C₀-C₂alkyl, (heterocycloalkyl)C₀-C₂alkyl, (heteroaryl)C₀-C₂alkyl, or (aryl)C₀-C₂alkyl;

at least two of Ca, Cb, and Cd are each independently chosen from ¹²C and ¹³C; and

n is an integer from 1 to 4.

In another aspect, the present invention is directed to a method of diagnosing or monitoring a patient suffering from cancer, the method comprising (1) administering a pharmaceutical composition comprising an effective amount of an active agent, wherein the active agent is the compound of Formula I, a pharmaceutically acceptable salt, or a combination thereof, together with a pharmaceutically acceptable carrier to the patient; and (2) diagnosing or monitoring the patient by hyperpolarized¹³C-MRI.

In another aspect, the present invention is directed to a process of synthesizing 1-¹³C-5-¹²C-diacid comprising treating a ¹³C-morpholine amide, an amine oxide, 3,4,5-trimethyl thiazolium iodide, and a ¹²C-michael acceptor to afford a 1-¹³C-5-¹²C-tricarbonyl compound, converting the tricarbonyl compound to a 1-¹³C-5-¹²C-diester, and hydrolyzing the corresponding diester to a corresponding 1-¹³C-5-¹²C-diacid.

These and other aspects will become apparent from the following detailed description and drawings.

### BRIEF DESCRIPTION OF THE DRAWINGS

The following Detailed Description, given by way of Examples, but not intended to limit the invention to specific embodiments described, may be understood in conjunction with the accompanying figures, in which:

FIG. 1 shows that the ¹²C enrichment of C5 on α-KG eliminated the peak contamination from the naturally-occurring [5-¹³C]-α-KG resulting in clean detection of [1 ¹³C]-2-HG via HP-¹³C-MRI;

FIG. 2 shows ¹³C signals derived from C5 of Sigma [1-¹³C]-α-KG are not detected in a phantom of hyperpolarized IPDC [1-¹³C-5-¹²C]-α-KG;

FIG. 3 shows the NMR spectra for in vitro detection of hyperpolarized [1 13C-5-¹²C]-α-KG and its metabolism by Hypersense/preclinical 3T MRI;

FIG. 4 shows the NMR spectra for in cellulo detection of hyperpolarized [1 13C-5-¹²C]-α-KG and its metabolism by Hypersense/preclinical 3T MRI.

FIG. 5 shows a graph of ¹³C-chemical shift in parts per million (ppm) versus time in seconds illustrating that the use of diethyl ketoglutarate I nHCT116R132H Cells shows better permeability;

FIG. 6A shows a natural abundance C5 and C2 peaks for α-KG were detected as minor peaks at 184 p.p.m. and 208 p.p.m., respectively;

FIG. 6B shows mouse xenografts after a tail injection of hyperpolarized-[1 ¹³C]-α-KG into HCT116IDH1 R132H;

FIG. 6C shows comparison of xenografts injected with [1-¹³C]-α-KG to control;

FIG. 6D shows comparison of IDH 1 R132H xenografts with, or without, the addition of ¹³C labeled α-KG:

FIG. 7A shows comparison of relative metabolites in acceptor well for α-KG, DM-α-KG, and DE-α-KG using a parallel artificial membrane permeability assay (PAMPA) to measure the ability of a probe to cross an artificial membrane barrier;

FIG. 7B shows a HP-¹³C-MRI spectra showing two major peaks corresponding to diethyl-[1-¹³C]-α-KG (163 p.p.m.) and diethyl-[1-¹³C]-α-KG hydrate (174 p.p.m.);

FIG. 8A shows ¹³C-NMR spectra showing the largest peak corresponding to [1-¹³C]-α-KG (172 p.p.m.) and only small peaks remaining for DE-[1-¹³C]-α-KG (174 p.p.m. and 163 p.p.m. for DE-[1-¹³C]-α-KG hydrate and DE-[1-¹³C]-α-KG respectively:

FIG. 8B shows time-course tracing of ¹³C-NMR peaks illustrating differential signal decay of the glutamate and glutamine.

### DETAILED DESCRIPTION OF THE INVENTION

### Terminology

Compounds are described using standard nomenclature. Unless defined otherwise, all technical and scientific terms used herein have the same meaning as is commonly understood by one of skill in the art to which this invention belongs.

The terms “a” and “an” do not denote a limitation of quantity, but rather denote the presence of at least one of the referenced items. The term “or” means “and/or.” The terms “comprising,” “having,” “including,” and “containing” are to be construed as open-ended terms (i.e., meaning “including, but not limited to”).

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

All methods described herein can be performed in any suitable order unless otherwise indicated herein or otherwise clearly contradicted by context. The use of any and all examples, or exemplary language (e.g., “such as”), is intended for illustration and does not pose a limitation on the scope of the disclosure unless otherwise claimed. No language in the specification should be construed as indicating any non-claimed element as essential to the practice of the invention. Unless defined otherwise, technical and scientific terms used herein have the same meaning as is commonly understood by one of skill in the art of this disclosure.

Furthermore, the disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

All compounds are understood to include all possible isotopes of atoms occurring in the compounds. Isotopes include those atoms having the same atomic number but different mass numbers. By way of general example, and without limitation, isotopes of hydrogen include tritium and deuterium and isotopes of carbon include ¹¹C, ¹³C, and ¹⁴C.

The opened ended term “comprising” includes the intermediate and closed terms “consisting essentially of” and “consisting of.”

The term “substituted” means that any one or more hydrogens on the designated atom or group is replaced with a selection from the indicated group, provided that the designated atom's normal valence is not exceeded. When the substituent is oxo (i.e., ═O), then 2 hydrogens on the atom are replaced. When aromatic moieties are substituted by an oxo group, the aromatic ring is replaced by the corresponding partially unsaturated ring. For example, a pyridyl group substituted by oxo is a pyridone. Combinations of substituents and/or variables are permissible only if such combinations result in stable compounds or useful synthetic intermediates. A stable compound or stable structure is meant to imply a compound that is sufficiently robust to survive isolation from a reaction mixture, and subsequent formulation into an effective therapeutic agent.

Suitable groups that may be present on an “optionally substituted” position include, but are not limited to, e.g., halogen, cyano, hydroxyl, amino, nitro, oxo, azido, alkanoyl (such as a C₂-C₆ alkanoyl group such as acyl or the like (—(C═O)alkyl)); carboxamido; alkylcarboxamide; alkyl groups, alkoxy groups, alkylthio groups including those having one or more thioether linkages, alkylsulfinyl groups including those having one or more sulfinyl linkages, alkylsulfonyl groups including those having one or more sulfonyl linkages, mono— and di-aminoalkyl groups including groups having one or more N atoms, all of the foregoing optional alkyl substituents may have one or more methylene groups replaced by an oxygen or —NH—, and have from about 1 to about 8, from about 1 to about 6, or from 1 to about 4 carbon atoms, cycloalkyl; phenyl; phenylalkyl with benzyl being an exemplary phenylalkyl group, phenylalkoxy with benzyloxy being an exemplary phenylalkoxy group. Alkylthio and alkoxy groups are attached to the position they substitute by the sulfur or oxygen atom respectively.

A dash (“-”) and (“ [symbol] ”) that is not between two letters or symbols is used to indicate a point of attachment for a substituent.

“Alkyl” includes both branched and straight chain saturated aliphatic hydrocarbon groups, having the specified number of carbon atoms, generally from 1 to about 8 carbon atoms. The term C₁-C₆alkyl as used herein indicates an alkyl group having from 1, 2, 3, 4, 5, or 6 carbon atoms. Other embodiments include alkyl groups having from 1 to 8 carbon atoms, 1 to 4 carbon atoms or 1 or 2 carbon atoms, e.g. C₁-C₈alkyl, C₁-C₄alkyl, and C₁-C₂alkyl. When C₀-Cₙ alkyl is used herein in conjunction with another group, for example,-C₀-C₂alkyl(phenyl), the indicated group, in this case phenyl, is either directly bound by a single covalent bond (C₀alkyl), or attached by an alkyl chain having the specified number of carbon atoms, in this case 1, 2, 3, or 4 carbon atoms. Alkyls can also be attached via other groups such as hetematomsas in —O—C₀-C₄alkyl(C₃-C₇cycloalkyl). Examples of alkyl include, but are not limited to, methyl, ethyl, n-propyl, isopropyl, n-butyl, 3-methylbutyl, t-butyl, n-pentyl, and sec-pentyl.

“Alkenyl” is a branched or straight chain aliphatic hydrocarbon group having one or more carbon-carbon double bonds that may occur at any stable point along the chain, having the specified number of carbon atoms. Examples of alkenyl include, but are not limited to, ethenyl and propenyl.

“Alkynyl” is a branched or straight chain aliphatic hydrocarbon group having one or more double carbon-carbon triple bonds that may occur at any stable point along the chain, having the specified number of carbon atoms.

“Alkoxy” is an alkyl group as defined above with the indicated number of carbon atoms covalently bound to the group it substitutes by an oxygen bridge (—O—). Examples of alkoxy include, but are not limited to, methoxy, ethoxy, n-propoxy, i-propoxy, n-butoxy, 2-butoxy, t-butoxy, n-pentoxy, 2-pentoxy, 3- pentoxy, isopentoxy, neopentoxy, n-hexoxy, 2-hexoxy, 3-hexoxy, and 3- methylpentoxy. Similarly, an “Alkylthio” or a “thioalkyl” group is an alkyl group as defined above with the indicated number of carbon atoms covalently bound to the group it substitutes by a sulfur bridge (—S—).

“Aryl” is a substituted stable monocyclic or polycyclic aromatic ring having 1 to 60 ring carbon atoms. Aryl groups include, but are not limited to, tolyl, xylyl, naphthyl, phenanthryl, and anthracenyl.

“Cycloalkyl” is a saturated hydrocarbon ring group, having the specified number of carbon atoms, usually from 3 to about 7 carbon atoms. Examples of cycloalkyl groups include cyclopropyl, cyclobutyl, cyclopentyl, or cyclohexyl as well as bridged or caged saturated ring groups such as norborane or adamantane. “—(C₀-Cₙalkyl)cycloalkyl” is a cycloalkyl group attached to the position it substitutes either by a single covalent bond (C₀) or by an alkylene linker having 1 to n carbon atoms.

“Halo” or “halogen” means iluom, chloro, bromo, or iodo.

“Heteroaryl” is a stable monocyclic aromatic ring having the indicated number of ring atoms which contains from 1 to 3, or in some embodiments from 1 to 2, heteroatoms chosen from N, O, and S, with remaining ring atoms being carbon, or a stable bicyclic or tricyclic system containing at least one 5- to 7-membered aromatic ring which contains from 1 to 3, or in some embodiments from 1 to 2, heteroatoms chosen from N, O, and S, with remaining ring atoms being carbon. Monocyclic heteroaryl groups typically have from 5 to 7 ring atoms. In some embodiments bicyclic heteroaryl groups are 9- to 10-membered heteroaryl groups, that is, groups containing 9 or 10 ring atoms in which one 5- to 7-member aromatic ring is fused to a second aromatic or non-aromatic ring. When the total number of S and O atoms in the heteroaryl group exceeds 1, these heteroatoms are not adjacent to one another. It is preferred that the total number of S and O atoms in the heteroaryl group is not more than 2. It is particularly preferred that the total number of S and O atoms in the aromatic heterocycle is not more than 1. Heteroaryl groups include, but are not limited to, oxazolyl, piperazinyl, pyranyl, pyrazinyl, pyrazolopyrimidinyl, pyrazolyl, pyridizinyl, pyridyl, pyrimidinyl, pyrrolyl, quinolinyl, tetrazolyl, thiazolyl, thienylpyrazolyl, thiophenyl, triazolyl, benzo[d]oxazolyl, benzofuranyl, benzothiazolyl, benzothiophenyl, benzoxadiazolyl, dihydrobenzodioxynyl, furanyl, imidazolyl, indolyl, isothiazolyl, and isoxazolyl.

“Heterocycle” is a saturated, unsaturated, or aromatic cyclic group having the indicated number of ring atoms containing from 1 to about 3 heteroatoms chosen from N, O, and S, with remaining ring atoms being carbon. Examples of heterocycle groups include piperazine and thiazole groups.

“Heterocycloalkyl” is a saturated cyclic group having the indicated number of ring atoms containing from 1 to about 3 heteroatoms chosen from N, O, and S, with remaining ring atoms being carbon. Examples of heterocycloalkyl groups include tetrahydrofuranyl and pyrrolidinyl groups.

“Haloalkyl” means both branched and straight-chain alkyl groups having the specified number of carbon atoms, substituted with 1 or more halogen atoms, generally up to the maximum allowable number of halogen atoms. Examples of haloalkyl include, but are not limited to, trifluoromethyl, difluoromethyl, 2-fluoroethyl, and penta-fluoroethyl.

“Haloalkoxy” is a haloalkyl group as defined above attached through an oxygen bridge (oxygen of an alcohol radical).

“Pharmaceutical compositions” means compositions comprising at least one active agent, such as a compound or salt of Formula I, and at least one other substance, such as a carrier. Pharmaceutical compositions meet the U.S. FDA's GMP (good manufacturing practice) standards for human or non-human drugs.

“Carrier” means a diluent, excipient, or vehicle with which an active compound is administered. A “pharmaceutically acceptable carrier” means a substance, e.g., excipient, diluent, or vehicle, that is useful in preparing a pharmaceutical composition that is generally safe, non-toxic and neither biologically nor otherwise undesirable, and includes a carrier that is acceptable for veterinary use as well as human pharmaceutical use. A “pharmaceutically acceptable carrier” includes both one and more than one such carrier.

A “patient” means a human or non-human animal in need of medical treatment. Medical treatment can include treatment of an existing condition, such as a disease or disorder or diagnostic treatment. In some embodiments the patient is a human patient.

“Providing” means giving, administering, selling, distributing, transferring (for profit or not), manufacturing, compounding, or dispensing.

“Treatment” or “treating” means providing an active compound to a patient in an amount sufficient to measurably reduce any cancer symptom, slow cancer progression or cause cancer regression. In certain embodiments treatment of the cancer may be commenced before the patient presents symptoms of the disease.

A “therapeutically effective amount” of a pharmaceutical composition means an amount effective, when administered to a patient, to provide a therapeutic benefit such as an amelioration of symptoms, decrease cancer progression, or cause cancer regression.

A significant change is any detectable change that is statistically significant in a standard parametric test of statistical significance such as Student's T-test, where p<0.05.

Chemical Description

Compounds of Formula I may contain one or more asymmetric elements such as stereogenic centers, stereogenic axes and the like, e.g., asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, all optical isomers in pure form and mixtures thereof are encompassed. In these situations, the single enantiomers, i.e., optically active forms can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column. All forms are contemplated herein regardless of the methods used to obtain them.

All forms (for example solvates, optical isomers, enantiomeric forms, tautomers, polymorphs, free compound and salts) of an active agent may be employed either alone or in combination.

The term “chiral” refers to molecules, which have the property of non-superimposability of the mirror image partner.

“Stereoisomers” are compounds, which have identical chemical constitution, but differ with regard to the arrangement of the atoms or groups in space.

A “diastereomer” is a stereoisomer with two or more centers of chirality and whose molecules are not mirror images of one another. Diastereomers have different physical properties, e.g., melting points, boiling points, spectral properties, and reactivities. Mixtures of diastereomers may separate under high resolution analytical procedures such as electrophoresis, crystallization in the presence of a resolving agent, or chromatography, using, for example a chiral HPLC column.

“Enantiomers” refer to two stereoisomers of a compound, which are non-superimposable mirror images of one another. A 50:50 mixture of enantiomers is referred to as a racemic mixture or a racemate, which may occur where there has been no stereoselection or stereospecificity in a chemical reaction or process.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory.

A “racemic mixture” or “racemate” is an equimolar (or 50:50) mixture of two enantiomeric species, devoid of optical activity. A racemic mixture may occur where there has been no stereoselection or stereospecificity in a chemical reaction or process.

“Tautomers” or “tautomeric forms” are constitutional isomers that readily interconvert, commonly by the migration of a hydrogen atom combined with a switch of a single bond and a double bond.

“Pharmaceutically acceptable salts” include derivatives of the disclosed compounds in which the parent compound is modified by making inorganic and organic, non toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media such as ether, ethyl acetate, ethanol, isopropanol, or acetonitrile are used, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like. Lists of additional suitable salts may be found, e.g., in G. Steffen Paulekuhn, et al., Journal of Medicinal Chemistry 2007, 50, 6665 and Handbook of Pharmaceutically Acceptable Salts: Properties, Selection and Use, P. Heinrich Stahl and Camille G. Wermuth Editor, Wiley-VCH, 2002.

The composition may further include at least one pharmaceutically acceptable excipient. A pharmaceutically acceptable excipient, as used herein, refers to a non-active pharmaceutical ingredient (“API”) substance such as a disintegrator, a binder, a filler, and a lubricant used in formulating pharmaceutical products. Each of these substances is generally safe for administering to humans according to established governmental standards, including those promulgated by the United States Food and Drug Administration (“FDA”).

A disintegrator, as used herein, refers to one or more of agar-agar, algins, calcium carbonate, carboxymethylcellulose, cellulose, clays, colloid silicon dioxide, croscarmellose sodium, crospovidone, gums, magnesium aluminium silicate, methylcellulose, polacrilin potassium, sodium alginate, low substituted hydmxypropylcellulose, and cross-linked polyvinylpyrmlidone hydroxypropylcellulose, sodium starch glycolate, and starch, but is not limited thereto.

A binder, as used herein, refers to one or more of microcrystalline cellulose, hydmxymethyl cellulose, and hydroxypropylcellulose, but is not limited thereto.

A filler, as used herein, refers to one or more of calcium carbonate, calcium phosphate, dibasic calcium phosphate, tribasic calcium sulfate, calcium carboxymethylcellulose, cellulose, dextrin derivatives, dextrin, dextrose, fructose, lactitol, lactose, magnesium carbonate, magnesium oxide, maltitol, maltodextrins, maltose, sorbitol, starch, sucrose, sugar, and xylitol, but is not limited thereto.

A lubricant, as used herein, refers to one or more of agar, calcium stearate, ethyl oleate, ethyl laureate, glycerin, glyceryl palmitostearate, hydrogenated vegetable oil, magnesium oxide, magnesium stearate, mannitol, poloxamer, glycols, sodium benzoate, sodium lauryl sulfate, sodium stearyl, sorbitol, stearic acid, talc, and zinc stearate, but is not limited thereto.

The composition according to the present invention may be administered to a patient by various routes. Examples of routes of administration include, but are not limited to, parenteral, e.g., intravenous, intradermal, subcutaneous, oral, intranasal (e.g., inhalation), transdermal (e.g., topical), transmucosal, and rectal administration. In an embodiment, the composition is formulated in accordance with routine procedures as a pharmaceutical composition adapted for intravenous, subcutaneous, intramuscular, oral, intranasal, or topical administration to human beings. Typically, compositions for intravenous administration are solutions in sterile isotonic aqueous buffer.

In accordance with any of the embodiments, the composition according to the present invention can be administered orally to a subject in need thereof. Formulations suitable for oral administration can consist of (a) liquid solutions, such as an effective amount of the compound dissolved in diluents, such as water, saline, or orange juice and include an additive, such as cyclodextrin (e.g., α—, β—, or γ-cyclodextrin, hydroxypmpyl cyclodextrin) or polyethylene glycol (e.g., PEG400); (b) capsules, sachets, tablets, lozenges, and troches, each containing a predetermined amount of the active ingredient, as solids or granules; (c) powders; (d) suspensions in an appropriate liquid; and (e) suitable emulsions and gels. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and cornstarch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such carriers as are known in the art.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The composition according to the present invention can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypmpylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene-polypropylene copolymers, (d) amphoteric detergents such as, for example, alkyl-beta-aminopropionates, and 2-alkyl-imidazoline quaternary ammonium salts, and (3) mixtures thereof.

The parenteral formulations will typically contain from about 0.5 to about 25% by weight of the composition according to the present invention in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

The composition according to the present invention may be made into injectable formulations. The requirements for effective pharmaceutical carriers for injectable compositions are well known to those of ordinary skill in the art. See Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986).

The composition according to the present invention may be administered in an effective amount. An “effective amount” means an amount sufficient to show a meaningful benefit in a patient. Effective amounts may vary depending upon the biological effect desired in a patient, condition to be treated, and/or the specific characteristics of the composition according to the present invention and the individual. In this respect, any suitable dose of the composition can be administered to the patient (e.g., human), according to the biological effect desired or the type of disease to be treated. Various general considerations taken into account in determining the “effective amount” are known to those of skill in the art and are described, e.g., in Gilman et al., eds., Goodman And Gilman's: The Pharmacological Bases of Therapeutics. 8th ed., Pergamon Press, 1990; and Remington's Pharmaceutical Sciences, 17th Ed., Mack Publishing Co., Easton, Pa., 1990, each of which is herein incorporated by reference. The dose of the composition according to the present invention desirably comprises about 0.1 mg per kilogram (kg) of the body weight of the patient (mg/kg) to about 400 mg/kg (for e.g., about 0.75 mg/kg, about 5 mg/kg, about 30 mg/kg, about 75 mg/kg, about 100 mg/kg, about 200 mg/kg, or about 300 mg/kg). In another embodiment, the dose of the composition according to the present invention comprises about 0.5 mg/kg to about 300 mg/kg (for e.g., about 0.75 mg/kg, about 5 mg/kg, about 50 mg/kg, about 100 mg/kg, or about 200 mg/kg), about 10 mg/kg to about 200 mg/kg (for e.g., about 25 mg/kg, about 75 mg/kg, or about 150 mg/kg), or about 50 mg/kg to about 100 mg/kg (for e.g., about 60 mg/kg, about 70 mg/kg, or about 90 mg/kg).

In an embodiment, the dose of the composition according to the present invention desirably comprises about 0.1 millimole (mmol) per kilogram (kg) of the body weight of the patient (nunol/kg) to about 10 mmol/kg (for e.g., about 0.1 mmol/kg, about 0.5 mmol/kg, about 1 mmol/kg, about 1.5 mmol/kg, about 2 mmol/kg/kg, about 2.5 mmol/kg/kg, about 3 mmol/kg, about 4 mmol/kg, about 5 mmol/kg, about 6 mmol/kg, about 7 mmol/kg, about 8 mmol/kg, about 9 mmol/kg, or about 10 mmol/kg).

As indicated above, the present invention is directed to a compound of Formula I

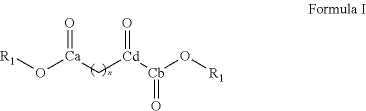

or a pharmaceutically acceptable salt thereof,

wherein in Formula I

R₁ is each independently selected from hydrogen, C₁-C₄alkyl, C₃-C₇cycloalkyl, (C₃-C₇cycloalkyl)C₀-C₂alkyl, (heterocycloalkyl)C₀-C₂alkyl, (hetemaryl)C₀-C₂alkyl, or (aryl)C₀-C₂alkyl;
at least two of Ca, Cb, and Cd are each independently chosen from ¹²C and ¹³C; and n is an integer from 1 to 4.

A compound of Formula I may be administered singularly (i.e., sole diagnosing or monitoring agent agent of a regime) to diagnose or monitor a patient suffering from cancer or may be administered in combination with another active agent. One or more compounds of Formula I may be administered in coordination with a regime of one or more other chemotherapeutic agents such as an antineoplastic drug, e.g., an alkylating agent (e.g., mechloroethamine, chlorambucil, cyclophosamide, melphalan, or ifosfamide), an antimetabolite such as a folate antagonist (e.g., methotrexate), a purine antagonist (e.g. 6-mercaptopurine) or a pyrimidine antagonist (e.g., 5-fluorouracil). Other, non-limiting examples of chemotherapeutic agents that might be used in coordination with one or more compounds of Formula 1, Formula 11, or Formula III include taxanes and topoisomerase inhibitors. In addition, other non-limiting examples of active therapeutics include biological agents, such as monoclonal antibodies or IgG chimeric molecules, that achieve their therapeutic effect by specifically binding to a receptor or ligand in a signal transduction pathway associated with cancer (e.g. therapeutic antibodies directed against CD20 (e.g. rituximab) or against VEGF (e.g. bevacizumab)).

Methods of diagnosing or monitoring provided herein are also useful for treatment of mammals other than humans, including for veterinary applications such as to treat horses and livestock e.g. cattle, sheep, cows, goats, swine and the like, and pets (companion animals) such as dogs and cats.

For diagnostic or research applications, a wide variety of mammals will be suitable subjects including rodents (e.g. mice, rats, hamsters), rabbits, primates and swine such as inbred pigs and the like. Additionally, for in vitro applications, such as in vitro diagnostic and research applications, body fluids (e.g., blood, plasma, serum, cellular interstitial fluid, saliva, feces and urine) and cell and tissue samples of the above subjects will be suitable for use.

Methods of diagnosing or monitoring include providing certain dosage amounts of an active agent to a patient. Dosage levels of each active agent of from about 0.1 millimole (mmol) per kilogram (kg) of the body weight of the patient (mmol/kg) to about 10 mmol/kg per day are useful in the methods of diagnosing or monitoring (for e.g., about 0.1 mmol/kg, about 0.5 mmol/kg, about 1 mmol/kg, about 1.5 mmol/kg, about 2 mmol/kg/kg, about 2.5 mmol/kg/kg, about 3 mmol/kg, about 4 mmol/kg, about 5 mmol/kg, about 6 mmol/kg, about 7 mmol/kg, about 8 mmol/kg, about 9 mmol/kg, or about 10 mmol/kg).

Dosage levels of each active agent of from about 0.1 mg to about 140 mg per kilogram of body weight per day are useful in the methods of diagnosing or monitoring (about 0.5 mg to about 7 g per patient per day). The amount of compound that may be combined with the carrier materials to produce a single dosage form will vary depending upon the patient treated and the particular mode of administration. Dosage unit forms will generally contain between from about 1 mg to about 500 mg of each active compound. In certain embodiments 25 mg to 500 mg, or 25 mg to 200 mg of the active agents are provided daily to a patient. Frequency of dosage may also vary depending on the compound used and the particular diagnosing or monitoring methods used. However, for most diagnosing or monitoring methods, a dosage regimen of 4 times daily or less can be used and in certain embodiments a dosage regimen of 1 or 2 times daily is used.

It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease undergoing therapy.

In an embodiment, the invention provides a compound of Formula i

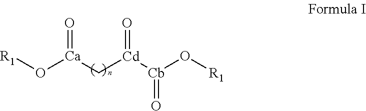

or a pharmaceutically acceptable salt thereof,

wherein in Formula I

R₁ is each independently selected from hydrogen, C₁-C₆alkyl, C₃-C₇cycloalkyl, (C₃-C₇cycloalkyl)C₀-C₂alkyl, (heterocycloalkyl)C₀-C₂alkyl, (hetemaryl)C₀-C₂alkyl, or (aryl)C₀-C₂alkyl;

at least two of Ca, Cb, and Cd are each independently chosen from ¹²C and ¹³C; and

n is an integer from 1 to 4.
In an embodiment, the compound or a pharmaceutically acceptable salt of Formula I,
wherein R₁ is each independently selected from hydrogen or C₁-C₆alkyl;
at least two of Ca, Cb, and Cd are each independently chosen from ¹²C and ¹³C; and
n is 2.
In an embodiment, the compound or a pharmaceutically acceptable salt of

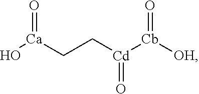

wherein at least two of Ca, Cb, and Cd are each independently chosen from
¹²C and ¹³C, and
¹²C is of about 90% to about 99.9% isotope abundance, preferably of about 99% isotope abundance, and more probably of about 99.9% isotope abundance. ¹³C is of about 80% to about 99.9% isotope abundance, preferably of about 99% isotope abundance, and more probably of about 99.9% isotope abundance.
In an embodiment, the compound or a pharmaceutically acceptable salt of Formula I is one of the following compounds:
diethyl-1-¹³C-5-¹²C-alpha ketoglutarate;
1-¹³C-5-¹²C-alpha ketoglutarate;
diethyl-1-¹³C-5-¹²C-2-hydroxyglutarate;
1-¹³C-5- ¹²C-2-hydroxyglutarate;
diethyl-1-¹³C-5-¹²C-glutamate;
1-¹³C-5-¹²C-glutamic acid;
1-¹²C-5¹³C-alpha ketoglutarate;
diethyl-1-¹²C-5-¹³C-alpha ketoglutarate;
1-¹³C-2-¹²C-5-¹²C-alpha ketoglutarate;
diethyl-1-¹³C-2-¹²C-5-¹²C-alpha ketoglutarate;
1-¹³C-2-¹³C-5-¹²C-alpha ketoglutarate; or
diethyl-1-¹³C-2-¹³C-5-¹²C-alpha ketoglutarate.

In an embodiment, a pharmaceutical composition comprising the compound or pharmaceutically acceptable salt of a compound of Formula I and a pharmaceutically acceptable carrier.

In an embodiment, the pharmaceutically acceptable carrier is selected from one or more of a sterile liquid or mixture of liquids, including water, saline, aqueous dextrose and related sugar solutions, an alcohol, ethers, an oil, a fatty acid, a fatty acid ester or glyceride, or an acetylated fatty acid glyceride with or without the addition of a pharmaceutically acceptable surfactant, suspending agent, carbomers, methylcellulose, hydroxypropylmethylcellulose, or carboxymethylcellulose, or emulsifying agents and other pharmaceutical adjuvants.

In an embodiment, the pharmaceutical composition further includes a pharmaceutically acceptable carrier and a regime of one or more other chemotherapeutic agents comprising an antineoplastic drug, an antimetabolite, a purine antagonist, a pyrimidine antagonist, taxanes and topoisomerase inhibitors, or biological agents.

In an embodiment, the invention provides a method of diagnosing or monitoring a patient suffering from cancer, the method including administering a pharmaceutical composition comprising an effective amount of an active agent, wherein the active agent is the compound of Formula I, a pharmaceutically acceptable salt, or a combination thereof, together with a pharmaceutically acceptable carrier to the patient; and diagnosing or monitoring the patient by hyperpolarized ¹³C-MRI. The active agent provided herein may be administered alone, or in combination with one or more other active agent.

In an embodiment, the active agent is a hyperpolarized active agent.

In an embodiment, the method includes identifying whether the patient has an IDH1 mutation.

In an embodiment, the patient is with types of cancer known to have IDH1 mutations.

In an embodiment, the method includes a tumor heterogeneity imaging comprising regions of heterogeneity within a single tumor or molecular differences among tumor cell populations at different anatomic locations.

In an embodiment, the method includes predictive imaging identification of therapies targeting an IDH1 pathway.

In an embodiment, the method includes monitoring a response of patient's tumor(s) to therapies.

In an embodiment, the method includes conducting a radiation treatment planning-imaging study with the compound or a pharmaceutically acceptable salt of any of the claims 1-4 fused with a traditional radiation planning CT or MRI scan.

In an embodiment, the method includes a noninvasive molecular tumor evolution monitoring for molecular changes.

In an embodiment, the method includes not exposing the patient to an ionizing radiation.

In an embodiment, the method includes administering the pharmaceutical composition which further includes a pharmaceutically acceptable carrier and a regime of one or more other chemotherapeutic agents comprising an antineoplastic drug, an antimetabolite, a purine antagonist, a pyrimidine antagonist, taxanes and topoisomerase inhibitors, or biological agents.

In an embodiment, a patient may be a human.

Hereinafter, an embodiment will be described in detail with reference to the following examples and procedures. However, these examples are not intended to limit the purpose and scope of the one or more example embodiments.

### EXAMPLES

### Abbreviations

ACN Acetonitrile
APCI Atmospheric pressure chemical ionization
DIC N,N′-Diisopropylcarbodiimide
DMEM Dulbecco's Modified Eagle Medium
DMF Dimethylformamide
DMSO Dimethylsulfoxide
EDC 1-Ethyl-3—(3-dimethylaminopropyl)carbodiimide
EST Electrospray Ionization
HRMS High-resolution mass spectrometry
HPLC High Performance Liquid Chromatography
L Liter
LCMS Liquid Chromatography/Mass Spectrometry
MHz Megahertz
MSD Mass Selective Detector
MRI Magnetic Resonance imaging
μL microliters
ml/mL milliliters
mg milligrams
mm millimeters
mM millimolar
mmol millimoles
MS Mass spectrometry
NAC [1-¹³C] N-Acetyl Cysteine
ng Nanogram
NHS N-Hydroxysuccinimide
NMR Nuclear Magnetic Resonance
THE Tetrahydrofuran
TLC Thin Layer Chromatography
TFA Trifluoroacetic acid
UV-VIS Ultraviolet-visible

General Methods

All organic precursors and solvents were obtained from commercial sources and used as received unless overwise noted. Labelled and deuterated staring materials were obtained from Cambridge Isotope Laboratories, Inc (Cambridge, Mass.). Thin-Layer Chromatography (TLC) analyses were performed with Analtech (Newark, Del.) silica gel GHLF 0.25 mm plates using UV and iodine detection. Flash chromatography was performed on a Teledyne Isco CombiFlash Companion instrument with UV detection at 254 nm. Analytical and/or preparative HPLC analysis were performed on an Agilent 1200 Series instruments equipped with multi wavelength detectors using an Agilent Prep-C18 column (4.6×50 mm, 5 μm) with a flow rate of 1 mL/min. Solvent A was 0.1% TFA in water, Solvent B was 0.1% TFA in ACN, and a linear gradient of 5% B to 85% B over 15 min was used. ESI and/or APCI mass spectrometry (ESI MS and/or APCI MS) were performed on LC/MSD TrapXC1 Agilent Technologies instrument. ESI and/or APCI mass spectrometry (ESI MS and/or APCI MS) were performed on 6130 Quadrupole LCMS Agilent Technologies instrument equipped with diode array detector. HRMS data was acquired on Waters XEVO G2-XS Q-Tof in ESI Positive mode. ¹H-NMR and ¹³C-NMR spectra were recorded with a Varian spectrometer operating at 400 MHz and 101 MHz respectively. Chemical shifts are reported in parts per million (δ) and are referenced to tetramethylsilane (TMS). UV-VIS spectrophotometry was carried out on a Perkin Elmer Lambda 25 Spectrometer.

2-HG, α-KG, L-glutamate (Glu), 1-¹³C-α-KG and N-acetyl-glutamine (NAG), ammonium acetate, ammonium hydroxide and UPLC/MS grade acetonitrile were purchased from Sigma-Aldrich (St. Louis, Mo.). Water was purified through a Milli-Q Integral 5 system supplied by EMD Millipore (Billerica, Mass.).

It is understood that the synthetic procedures described herein can be performed with any isotopic form of carbon, for example, ¹²C, ¹³C or ¹⁴C, at any position of the final product or any of the reagents used for the synthesis.

Cell Culture and Animal Studies

All of the animal experiments were conducted in compliance with the Guide for the Care and Use of Laboratory Animal Resources, and experimental protocols were approved by the Animal Care and Use Committee, National Cancer institute (NCI-CCR-ACUC). HCT116 IDH 1 wild type and mutant cells were purchased from Horizon Discovery (Cambridge, UK). Cells were cultured at 37° C. under 5% CO₂ in RPMI medium supplemented with 10% FBS, 100 U/mL of penicillin, and 100 μg/mL of streptomycin.

Reagent Preparation

All α-KG derivatives were dissolved in mixture of D₂O: D-glycerol=1:1 containing 17.3 mM of Ox 063 at a concentration of 5.9 molar (M). After three times of freeze and thaw cycle using liquid nitrogen followed by vortex for 1 min, samples were stored at 4° C. until use. ¹³C MRI of hyperpolarized ¹³C-labeled α-KG metabolism

For all hyperpolarization experiments, 35 μL of Sigma [1-¹³C]-α-KG, [1-¹³C-5-¹²C]—,—α-KG, or [2-¹³C]-α-KG solution containing 2.5 mM of the gadolium chelate (PmHance, Bracco Diagnostics, Milano, Italy) were polarized at 3.35 T and 1.45-1.5 K in the Hypersense DNP polarizer (Oxford Instruments, Abingdon, UK) for 3-5 hour (h) according to manufacturer's instructions. The polarized samples were rapidly dissolved in 4.0 ml of alkaline buffer containing 25 mM Tris(hydmxymethyl)aminomethane, 50 mg/L ethylendiaminetetraacetic acid, and 37.5 mM NaOH, for the final dissolution buffer to be pH 7.4 after mixture with α-KG. For in vitro experiments, the hyperpolarized ¹³C-α-KG solution (1 mL) was injected into a glass vial (Wheaton® 224882 Lab File™ 4 mL Clear Glass Sample Vials for Aqueous Samples, Wheaton Science Products, Millville, NJ) placed in a 3T scanner (MR Solutions, Guildford, UK) via a plastic tube using a 17-mm custom-build ¹³C solenoid leg coil placed inside of a saddle coil for ¹H.

### Preparation of cells for in vitro HP-¹³C-MRI

Cells were cultured for 24 hours (h) after plating and harvested by trypsinization. Cells were washed with serum-free DMEM without pyruvate, counted, and resuspended in serum-free DMEM without pyruvate with the concentration of 5.0×10′ cells/mL. After pre-warmed to 37° C., 2 mL of cell suspension (1.0×10⁸ cells total) were transferred to glass vial immediately before measurement.

Animal Experiments

2.0×10⁶ cells were subcutaneously injected into right hind legs of nude mice (Nu/Nu). In vivo measurements were done with the tumors>1,000 mm³ in size, measured by caliper. For injection of hyperpolarized ¹³C compounds, reagents were injected into tail vain through thin tube equipped in 3T scanner. For extraction of metabolites after HP-¹³C-MRI experiments, tumors were treated in reference to previous reports with slight modifications. Ivanisevic, J., et al., Toward'omic scale metabolite profiling: a dual separation-mass spectrometry approach for coverage of lipid and central carbon metabolism. Anal Chem, 2013. 85(14): p. 6876-84; Masson, P., et al., Optimization and evaluation of metabolite extraction protocols for untargeted metabolic profiling of liver samples by UPLC-MS. Anal Chem, 2010. 82(18): p. 7779-86; Vorkas, P. A., et al., Untargeted UPLC-MS profiling pipeline to expand tissue metabolome coverage: application to cardiovascular disease. Anal Chem, 2015. 87(8): p. 4184-93. After harvesting, tumors were immediately cut into pieces and flash-frozen in liquid nitrogen and stored at −80° C. until use.

### Preparation of Tumor Extract for MS Analysis

Tumor extractions were performed following published procedures. Kishimoto, S., et al., Imaging of glucose metabolism by ¹³C-MRI distinguishes pancreatic cancer subtypes in mice. Elife, 2019. 8: p. e46312. Crooks, D. R., T. W. Fan, and W. M. Linehan. Metabolic Labeling of Cultured Mammalian Cells for Stable Isotope-Resolved Metabolomics: Practical Aspects of Tissue Culture and Sample Extraction. Methods Mol Biol, 2019. 1928: p. 1-27. Briefly, 50 mg of diethyl 1-¹³C-5-¹²C-α-KG was injected intravenously through the tail vein of a nude mouse bearing a HCT116 IDH 1 R132H xenograft to start the labelling experiment when the tumor size reached approximately 1250 mm³. Mice were sacrificed by cervical dislocation 10 minutes after injection, following an intravenous saline flush. The tumor was then excised and immediately snap frozen in liquid nitrogen and then stored at −80° C. until the extraction procedure. The polar fractions were isolated from the frozen tumor sections using a modification of a previously published procedure for cell extracts. Id. Briefly, a section of the frozen tumor was cut and then pulverized in liquid nitrogen using a cryogenic grinder (Freezer/Mill 6875, Spex SamplePrep). Approximately 50 mg aliquots of the ground tissue powder were weighed and then immediately quenched with 2 ml of acetonitrile at 20° C. The solution was allowed to thaw on ice and 1.5 ml of ice cold dd H₂O was added to the thawed extract. Lipids and non-polar metabolites were extracted by the addition of 1 ml of −20° C. chloroform with vigorous mixing. Addition of chloroform creates a three-phase system consisting of the polar and nonpolar fractions and an interphase layer consisting primarily of proteins. Following centrifugation at 6400 g for 30 min, 90% of the aqueous phase was transferred to a pre-tared microcentrifuge tube. After removal of the non-polar chloroform phase, the interphase layer was washed with ice cold 2:1 choloroform/methanol containing 1 mM BHT and recentrifuged. The aqueous phases were then combined and lyophilized. To remove residual proteins, the lyophilized powder was reconstituted in 100 μL of ice cold dd H₂O followed by 400 μL of ice-cold acetone. Samples were then incubated at 80° C. for 30 minutes to facilitate protein precipitation. The protein precipitate was isolated by centrifugation for 30 minutes at 14,000 rpm. The pellet was then washed with 100 μL of 60% acetonitrile and the remaining supernatant after centrifugation re-lyophilized before analysis by MS.

Cell Permeability Assay

Unlabeled α-KG, dimethyl-α-KG, and diethyl-α-KG permeability was measured using a PAMPA assay (R&D Systems). α-KG and derivatives were added to donor 96-well plates and allowed to pass through a dodecane membrane supplemented with 2% lectin for 24 hours at 37° C. Concentrations of α-KG and derivatives from the filtrate solution were assayed by a ultraviolet-visible spectrophotometer (BioTek™ Synergy™ H1 Hybrid Multi-Mode Mont chrotnator Fluorescence Microplate Reader) at 270 μM and compared to a respective standard curve for each α-KG derivative.

Instrumentation for LC-MS/MS Analysis

LC-MS/MS analysis was performed on an Agilent 6460C triple quadrupole mass spectrometer with an ESI source. The LC inlet was an Agilent 1200 series chromatographic system equipped with 1260 binary pump, 1290 thermostatted column compartment and 1260 high performance autosampler. Instrument control and data processing was performed using Agilent's MassHunter Software.

Chromatographic Conditions

Metabolites were measured by the method with minor modifications of previous reports. Xia, J., et al., MetaboAnalyst 3.0—making metabolomics more meaningful. Nucleic Acids Res, 2015. 43(W 1): p. W251-7. Xia, J. and D. S. Wishart, Using Metabo Analyst 3.0 for Comprehensive Metabolomics Data Analysis. Curr Protoc Bioinformatics, 2016. 55: p. 14 10 1-14 10 91.Yuan, M., et al., A positive/negative ion-switching, targeted mass spectrometry- based metabolomics platform for bodily fluids, cells, and fresh and fixed tissue. Nat Protoc, 2012. 7(5): p. 872-81. Chromatographic separations were carried out with gradient elution in hydrophilic interaction chromatography (HILIC) mode on Phenomenex Luna-NH2 (2.0×100 mm, 3.0 μm). The mobile phase was composed of aqueous buffer (Solvent A) and organic solvent (Solvent B) each containing 10 mM ammonium acetate with ammonium hydroxide added to adjust pH to 9.0. Solvent A contained a 95:5 mixture of water:acetonitrile, whereas solvent B contained a 95:5 mixture of acetonitrile:water. The analytes were eluted from the column by a linear gradient which started at 60% B, held at initial conditions for 1.0 min, then decreased from 60% to 5% B within 8 min and held at 5% B for 5.0 min then returned to the initial conditions. A 10-min equilibrium time between injections was used to ensure reproducible retention times. The flow rate was set at 0.5 mL/min. The column oven was kept at 40° C. throughout the analysis. The injection volume was 5 μL and the autosampler rack temperature was 8° C. The needle wash solvent was a mixture of 50:50 acetonitrile:water. Tumor samples were mixed with an equal volume of NAG (500 ng/mL in 50:50 acetonitrile:water) as an internal standard for monitoring system performance. A pooled tumor sample, prepared by combining 5 μL from each tumor sample, was used to condition the column before analysis of the actual samples. A QC sample consisting of 1 μg/mL of unlabeled standards (2-HG, α-KG) was used to determine retention times of the labeled compounds.

Mass Spectrometric conditions Mass spectrometric data were acquired in positive/negative ion switching mode with the following ESI-MS parameters: gas temperature 350° C.; gas flow 13 Umin; nebulizer 45 psi; capillary voltage 4000 V. Nitrogen was used as desolvation gas and collision gas, dwell time were set at 80 ms for each transition. Cell Accelerator Voltage was set to 7 and quantification was done in multiple reaction monitoring (MRM) mode. Precursor and product ion selection was determined experimentally using authentic samples when available. When authentic samples were not available, transitions were based on the unlabeled precursor and adjusted to incorporate the labeled atom.

Data Analysis

For LC-MS/MS data analysis, peak integration was performed in Agilent's MassHunter Software. Statistical analysis (fold-change and t-test) was performed in MetaboAnalyst 3.0 (www.metaboanalyst.ca) [36, 37]. No filtering or data normalization was performed before analysis. A t-test threshold of 0.05 was used to determine significant changes.

Statistics

Results were presented as means±SD (standard deviation) unless otherwise stated. Significance in differences were tested by Student's t-test or other methods stated in the text. Differences with p<0.05 was set to be significant.

### Example 1

### Synthesis of 2,5-Dioxopyrrolidin-1-yl 2-bromoacetate-1-¹³C

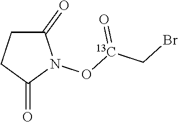 [SMILES: O=C1CCC(=O)N1O[13C](=O)CBr]

DIC (3.3 ml, 21.3 mmol) was added dropwise to a solution of 2-Bmmoacetic-1-¹³C acid (Cambridge Isotope Laboratories, 2.82 grams (g) 20.3 mmol) and N-hydroxysuccinamide (2.54 g, 22.3 mmol) in anhydrous isopropanol (30 ml) at room temperature. An exothermic reaction was observed, and white crystalline material formed. The resulting reaction mixture was stirred at room temperature for 1 h, followed by 16 h at 4° C. The solids were collected by filtration and washed thoroughly with cold isopropanol. The resulting colorless crystalline solid was dried in vacuo to give the desired product 2,5-Dioxopyrrolidin-1-yl 2-bromoacetate-1-¹³C and used in next step without purification. Yield: 4.38 g, 18.56 mmol, 91%. ¹H-NMR (400 MHz, CDCl₃) δ 4.10 (d, ¹³C—H coupling, 8 Hz, 2H) 2.87 (bs, 4H). ¹³C NMR (100 MHz, CDCl₃) δ 168.34, 162.92 (¹³C label), 25.56, 21.42-20.77 (d, ¹³C—C coupling, 65 Hz).

### Example 2

### Synthesis of 2-Bromo-1-morpholinoethan-1-one-1-¹³C (Compound 6)

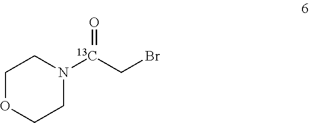

Morpholine (1.62 ml, 18.6 mmol) was added to a solution of 2,5-dioxopyrmlidin-1-yl 2-bromoacetate-1-¹³C (4.38 g, 18.6 mmol, NHS ester) in anhydrous acetonitrile (70 ml) at room temperature under argon. The resulting pale-yellow solution was stirred at room temperature for 1 h. LCMS of the reaction indicated complete consumption of the NHS ester and formation of desired product. The reaction mixture was concentrated, and the residue was purified by column chromatography on silica gel using 25 to 75% ethyl acetate in hexane to give the desired product, 2-Bromo-1-morpholinoethan-1-one-1-¹³C (Compound 6), as a colorless solid. Yield: 3.1 g, 14.9 mmol 80%. ¹H NMR (400 MHz) CDCl₃ 3.85 (d, 4 Hz¹³C—H Coupling, 2H), 3.73 (m. 2H), 3.69 (m. 2H′), 3.63 (m, 2H), 3.52 (m, 2H). ¹³C NMR (100 MHz, CDCl₃) δ 165.34 (¹³C label), 66.60 (d, ¹³C—C coupling, 1 Hz), 66.35 (d, ¹³C—C coupling, 1 Hz), 47.14 (d, ¹³C—C coupling, 2 Hz), 42.39, 25.65-25.08 (d, ¹³C—C coupling, 57 Hz).

HRMS M+H Calculated: 209.0010 Observed: 209.0011.

### Example 3

### Synthesis of Acrylic-1-¹²C acid

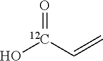

¹²C-enriched carbon dioxide was bubbled through a solution of vinylmagnesium chloride (1.6 M in THF, 100 ml) in anhydrous tetrahydrofuran (100 ml) at −78° C. (clear solution of vinlymagnesium chloride becomes turbid with precipitate). The ¹²C-enriched carbon dioxide (15 to 20 g) was bubbled into the yellow-brown solution at −78° C. until the color changes to pale yellow suspension. The reaction mixture was stirred at −78° C. for 2 h and then reaction quenched with concentrated hydrochloric acid (1.1 equivalents (eq.) 14 ml). Reaction mixture was then diluted with 100 ml diethyl ether and 100 ml water. The organic layer was separated, and the aqueous layer was extracted with diethyl ether (2×), the combined organic layer was dried over anhydrous sodium sulfate and concentrated in vacuo to give the desired product Acrylic-1-¹²C acid as a colorless oil, which was used in the next step without purification (9.2 g, 128 mmol, Yield 91%).

### Example 4

### Synthesis of Benzyl acrylate-1-¹²C (Compound 7)

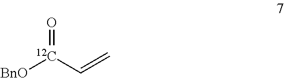

To a solution of EDCI.HCl (6.9 g, 36 mmol) in anhydrous dichloromethane (120 ml) under argon were added benzyl alcohol (3.1 ml, 30 mmol) and 4-dimethylaminopyridine (732 mg, 6 mmol) at 0° C. ¹²C-acrylic acid (3.24 g, 45 mmol) was slowly added dropwise to the solution over a period of 5 min, during the addition the solution developed a slight yellow color. The reaction was allowed to proceed overnight with warming to room temperature. The reaction was quenched by addition of saturated sodium bicarbonate solution, organic layer was separated, and aqueous layer was extracted with dichlorometane (2×). Combined organic layer was washed with 1 molar (M) hydrochloric acid (2×), water (1×) and brine (1×), finally organic layer was dried over anhydrous sodium sulfate and concentrated in vacuo. Residue was then chromatographed over silica-gel using 0 to 25% ethyl acetate in hexane to give desired product, Compound 7, as a colorless oil (2.16 g, 13.3 mmol, 44% for 2 steps). ¹H NMR (400 MHz, CDCl₃) δ7.36 (m, 5H), 6.48-6.43 (dd, J=20, 4 Hz, 1H), 6.21-6.14 (dd, J=20, 8 Hz, 1H), 5.87-5.84 (dd, J=8, 4 Hz, 1H), 5.21 (s, 2H). ¹³C NMR (100 MHz, CDCl₃) δ 135.86, 131.03, 128.55, 128.31, 128.24, 128.20, 66.29.

### Example 5

### Synthesis of Benzyl 5-morpholino-4,5-dioxopentanoate-1-¹²C-5-¹³C (Compound 8)

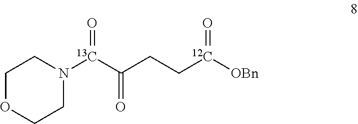 [SMILES: O=C(CC[12C](=O)OCc1ccccc1)[13C](=O)N1CCOCC1]

To a suspension 4-Dimethylaminopyridine N-Oxide hydrate (1.89 g, 13.68 mmol based on anhydrous weight) in anhydrous DMF (27 ml) was added solid 2-bromo-1-morpholinoethan-1-one-1-¹³C (Compound 6) (1.43 g, 6.84 mmol) in one portion under argon at room temperature. After the addition a clear solution was obtained. 3,4,5-trimethylthiazolium iodide (349 mg, 1.37 mmol) and Benzyl acrylate-1-¹²C (Compound 7) (2.13 ml, 13.68 mmol) were added to the solution. Finally, to the reaction mixture triethylamine (2.86 ml, 20.52 mmol) was added. Immediately, the reaction mixture developed a cherry red color, and was allowed to proceed at 50° C. for 18 h. After this time tlc and LCMS indicated complete consumption of starting material. The reaction was quenched by pouring into 90 ml 1 M hydrochloric acid. Aqueous layer was then extracted with ethyl acetate (3×), combined organic layer was then washed with water (1×) and brine (1×). Finally, organic layer was dried over anhydrous sodium sulfate, concentrated and chromatographed over silica gel using 25 to 75% ethyl acetate in hexane to give the desired product off white solid. Yield: 1.9 g, 6.2 mmol Yield 90% purity 95%. This product was then recrystallized by dissolving in 20 ml of hot ethanol and cooling to mom temperature gave an analytically pure product, Compound 8, as white needles (1.6 g). ¹H NMR (400 MHz, CDCl₃) δ 7.39-7.30 (m, 5H), 5.12 (s, 2H), 3.73-3.70 (m, 2H), 3.633.66-3.61 (m, 4H), 3.48-3.46 (m, 2H), 3.08-3.05 (m, 2H), 2.78-2.75 (m, 2H). ¹³C NMR (100 MHz, CDCl₃) δ 199.14-198.51 (d, ¹³C—C coupling, 63 Hz), 164.87 (¹³C label), 135.57, 128.59, 128.37, 128.18, 66.90-66.89 (d, ¹³C—C coupling, 1 Hz), 66.76, 66.53-66.52 (d, ¹³C—C coupling, 1 Hz), 46.05-46.04 (d, ¹³C—C coupling, 1 Hz), 41.86, 34.69-34.55 (d, ¹³C—C coupling, 14 Hz), 27.58-27.56 (d, ¹³C—C coupling, 2 Hz).

HRMS M+H Calculated: 307.1370 Observed: 307.1375.

### Example 6

### Synthesis of Diisopropyl 2-oxopentanedioate-5-¹²C-1-¹³C (Compound 10)

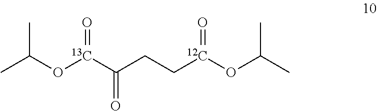 [SMILES: CC(C)O[12C](=O)CCC(=O)[13C](=O)OC(C)C]

To a solution of Benzyl 5-morpholino-4,5-dioxopentanoate-1-¹²C-5-¹³C (Compound 8) (1.9 g, 6.24 mmol) in anhydrous isopropanol (25 ml) was added concentrated sulfuric acid (2 ml, 37.45 mmol). The resulting reaction mixture was refluxed at 100° C. overnight after this time TLC and LCMS of the reaction indicated complete consumption of starting material. All the isopropanol was removed in vacuo and residue was taken in water and extracted with diethyl ether (3×). Combined organic layer was washed with saturated sodium bicarbonate (1×), water (1×) and brine (1×), finally organic layer was dried over anhydrous sodium sulfate and concentrated. The oily residue was then chromatographed over silica gel using 5 to 4.0% ethyl acetate in hexane to give desired product, Compound 10, as a colorless oil. Yield: 1.12 g, 4.84 mmol, 78%. ¹H NMR (400 MHz, CDCl₃) δ 5.14 (dtd, J=12.6, 6.3, 2.6 Hz, 1H), 4.99 (kept, J=6.3 Hz, 1H), 3.13 (t, J=6.5 Hz, 2H), 2.62 (t, J=6.6 Hz, 2H), 1.34 (d, J=6.3 Hz, 6H), 1.22 (d, J=6.3 Hz, 6H). ¹³C NMR (101 MHz, CDCl₃) δ193.04 (d, ¹³C—C coupling, 68 Hz), 160.18 (¹³C label), 70.76 (d, ¹³C—C coupling, 2 Hz), 68.31, 34.18 (d, ¹³C—C coupling, 16 Hz), 28.00 (d. ¹³C—C coupling, 1 Hz), 21.74, 21.56 (d, ¹³C—C coupling, 2 Hz).

HRMS M+H Calculated: 232.1261 Observed: 232.1271.

### Example 7

### Synthesis of Butyl-benzyl-alpha-ketoglutarate PGP-34,C₃

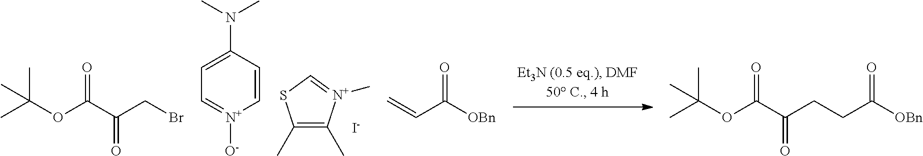

DMAP-oxide (166 mg, 1.2 mmol) and trimethylthiazolium iodide (51 mg, 0.2 mmol) were suspended in anhydrous dimethylformamide (4 ml) under argon. To the suspension was added t-Butyl bromoacetate (148 ul, 1 mmol) and stirred at rt until a clear solution was obtained (<5 min). Then to this solution were added benzyl acrylate (234 ul, 1.5 mmol) and triethylamine (70 ul, 0.5 mmol). The solution immediately developed cherry-red color and the reaction mixture was then heated to 50° C. for 4 h. During this time reaction color changed to pale yellow color, after 4 h reaction was quenched by addition of 15 ml water and extracted with EtOAc (3×10 ml). Combined organic layer was washed with water (1×) and brine (1×), finally organic layer was dried over anhydrous magnesium sulfate and concentrated in vacuo. Residue was chromatographed over 12 g prepacked silica gel column using 0-30% ethyl acetate in hexane to give desired product as colorless oil. Yield 135 mg (45%). Although the above scheme is show with reagents containing ¹⁴C, it is understood that this synthesis can be performed with any isotopic form of carbon, for example, ¹²C, ¹³C. or ¹⁴C, at any position of the final product or any of the reagents used for the synthesis.

### Example 8

Synthesis of Diethyl 2-oxopentanedioate-5-¹²C-1-¹³C (Compound 2)

Concentrated sulfuric acid (190 IL, 3.44 mmol) was added to a solution of ethyl 5-morpholino-4,5-dioxopentanoate-1-¹²C-5-¹³C (210 mg, 0.86 mmol) in ethyl alcohol (3 ml). The resulting reaction mixture was irradiated with microwaves for 2 h at 100° C. The solvent was removed in vacuo and residue was taken in diethyl ether and washed with saturated sodium bicarbonate (2 times), water (2 times), brine (1 time). Finally, the organic layer was dried over anhydrous sodium sulfate and concentrated to give the pure desired product as colorless oil. Yield 136 mg, 0.67 mmol, 78%. ¹H NMR (400 MHz, CDCl₃) δ 4.33 (qd, J=7.1, 3.0 Hz, H—H and ¹³C—H coupling, 2H), 4.14 (q, J=7.1 Hz, 2H), 3.15 (ddd, J=6.3, 0.4 Hz, 2H), 2.66 (t, J=6.5 Hz, 2H), 1.37 (t, J=7.1 Hz, 3H), 1.25 (t, J=7.1 Hz, 3H). ¹³C NMR (101 MHz, CDCl₃) δ 193.01-192.33 (d, ¹³C—C coupling, 68 Hz), 160.54 (¹³C label), 62.56-62.54 (d, ¹³C—C coupling, 2 Hz), 60.88, 34.22-34.06 (d, ¹³C—C coupling, 16 Hz), 27.69-27.67 (d, ¹³C—C coupling, 2 Hz), 14.12, 13.97-13.95 (d, ¹³C—C coupling, 2 Hz).

### Example 9

Synthesis of 2—Oxopentanedioic-5-¹²C-1-¹³C acid (Compound 11)

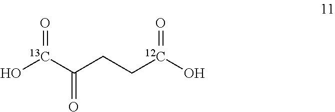

To Diisopropyl 2-oxopentanedioate-5-¹²C-1-¹³C (Compound 10) (0.1.5 g, 6.5 mmol) was added 25 ml of 3 M hydrochloric acid and the resulting biphasic reaction mixture was heated to reflux at 110° C. until the reaction becomes clear solution (˜3 h). After this tlc of the reaction indicated no starting material was left. Most of the acid was removed in vacuo at 35° C. and the residue was redissolved in water and lyophilized to give the desired product, Compound 11, as white solid which was supplied without any purification. Yield: 900 mg. 6.12 mmol, 95%. ¹H NMR (400 MHz, D₂O) δ2.91 (s), 2.59 (t, J=6.6 Hz), 2.35 (t, J=7.6 Hz), 2.03 (td. J=7.6, 3.3 Hz). ¹³C NMR (101 MHz, D₂O) δ174.09, 94.02-93.33 (d, ¹³C—C coupling, 69.69 Hz), 33.28-33.25 (d, ¹³C—C coupling, 3.03 Hz), 28.18 (major component), 27.53 (minor component).

HRMS M−H Calculated: 146.0176 Observed:146.0158.

### NMR and MS Analysis

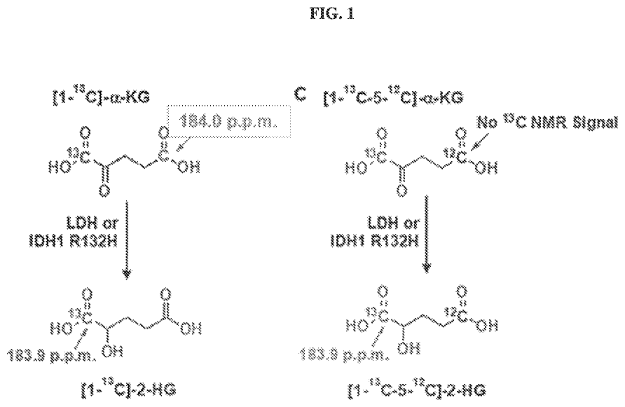

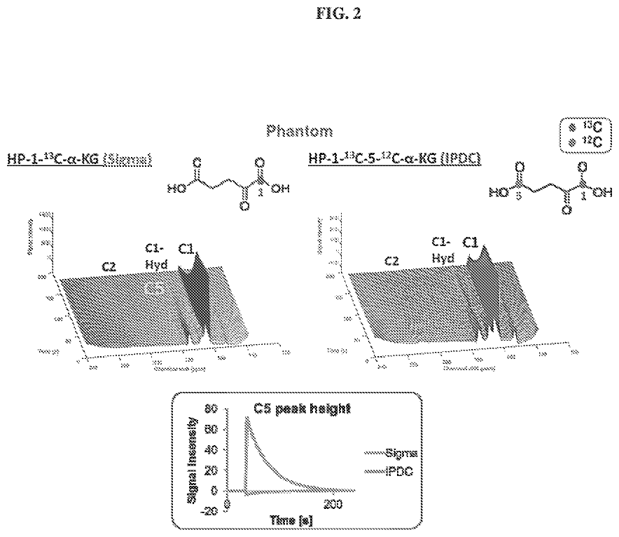

To determine whether [1-¹³C-5-¹²C]-α-KG would successfully eliminate the C5 peak in the HP-¹³C-MRI, signals from hyperpolarized [1-¹³C-]-α-KG and [1-¹³C-5-¹²C]-α-KG were compared. As shown in FIG. 1, ¹²C enrichment of C5 on α-KG eliminated the peak contamination from the naturally-occurring [5-¹³C]-α-KG and allowed for clean detection of [1-¹³C]-2-HG via HP-¹³C-MRI. As shown in FIG. 2, ¹³C signals derived from C5 of Sigma [1-¹³C]-α-KG are not detected in a phantom of hyperpolarized IPDC [1-¹³C-5-¹²C]-α-KG. The disappearance of this signal suggests that ¹²C enrichment of the C5 position of α-KG successfully eliminated the signal from the naturally abundant ¹³C at this position. In both [1-¹³C-]-α-KG and [1-¹³C-5-¹²C]-α-KG spectra, C1-hydrate, and C2 peaks were identified at 182 and 208 ppm, respectively.

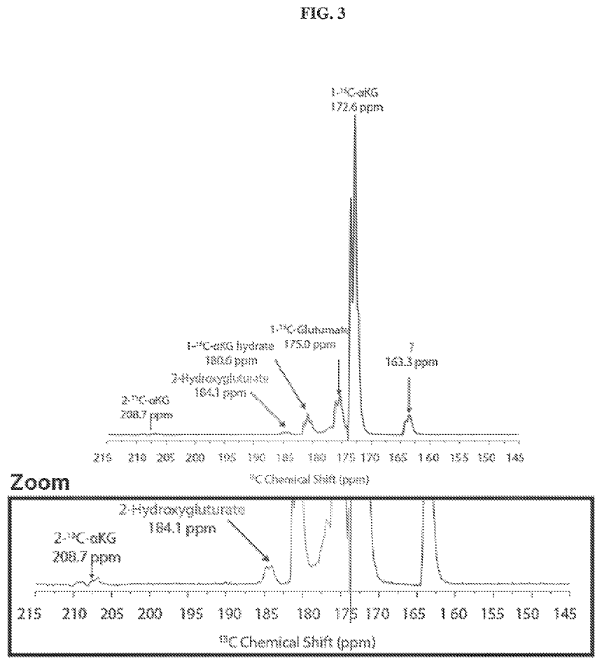

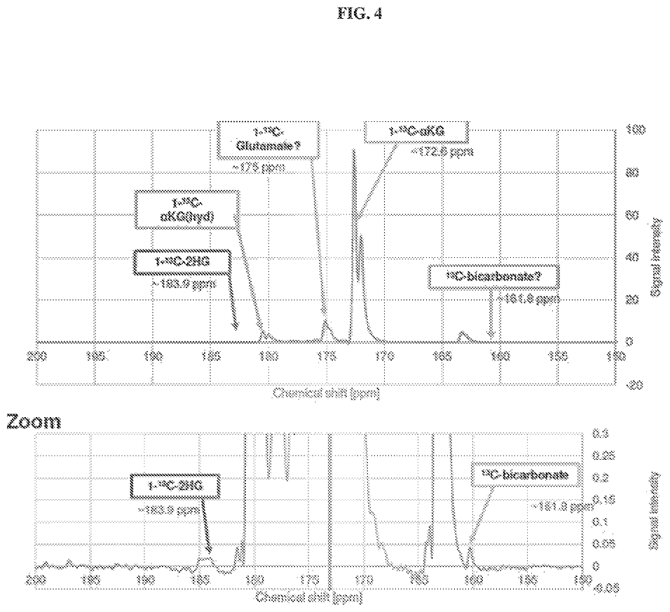

With the spectral signal corresponding to C5 of α-KG eliminated, the utility of [1-¹³C-5-¹²C]-α-KG as a probe to measure α-KG metabolism was tested. As lactate dehydrogenase can catalyze the conversion of α-KG to 2-HG via a non-canonical reaction, purified lactate dehydmgenase was utilized as a proof of principle for the use of [1-¹³C-5 ¹²C]-α-KG. As shown in FIG. 3, the NMR spectra for in vitro detection of hyperpolarized [1 13C-5-¹²C]-α-KG and its metabolism by Hypersense/preclinical 3T MRI shows the signal of [1-¹³C]-2-HG at 184.1 parts per million (ppm).

[1-¹³C-5-¹²-C]-α-KG allows for detection of [1-¹³C]-2-HG using LDH enzymes, this probe was tested in cellulo. The metabolism of α-KG in HCT116 IDH1 R132H cells was monitored by adding hyperpolarized [1-¹³C-5-¹²C]-α-KG directly before the HP-¹³C-MRI measurements. As shown in FIG. 4, the NMR spectra for in cellulo detection of hyperpolarized IPDC [1-¹³C-5-¹²C]-α-KG and its metabolism by Hypersense/preclinical 3T MRI shows the signal of [1-¹³C]-2-HG at 183.9 ppm. These results demonstrate [1-¹³C-5 ¹²C]-α-KG as a probe for non-invasive imaging of IDH1 status. By removing peak-contamination from the naturally-occurring [5-¹³C]-α-KG, the [1-¹³C]-2-HG produced in IDH1 mutant cells could be detected cleanly in cellulo using [1-¹³C-5-¹²C]-α-KG.

### Diethyl-[1-¹³C-5-¹²C]-α-KG (DE-[1-¹³C]-α-KG) is significantly more permeable than [1-¹³C-5¹²C]-α-KG (also mentioned as [1-¹³C]-α-KG)

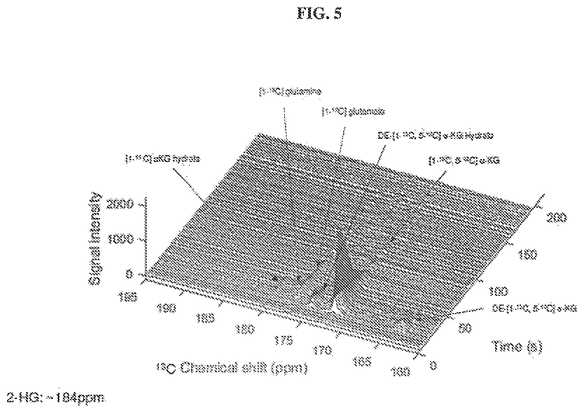

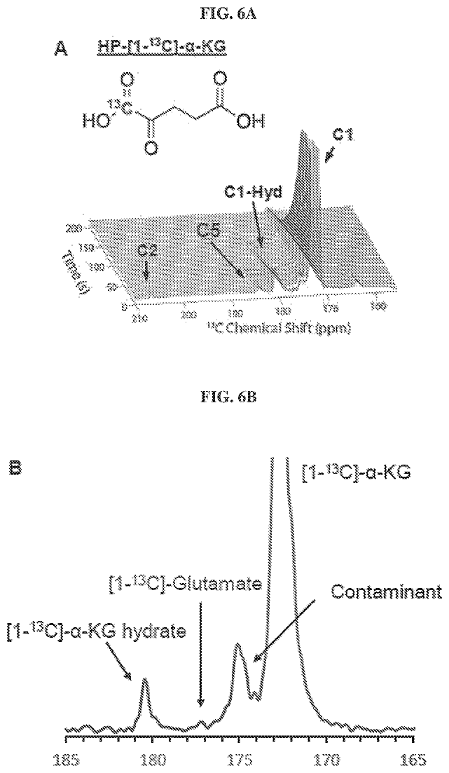

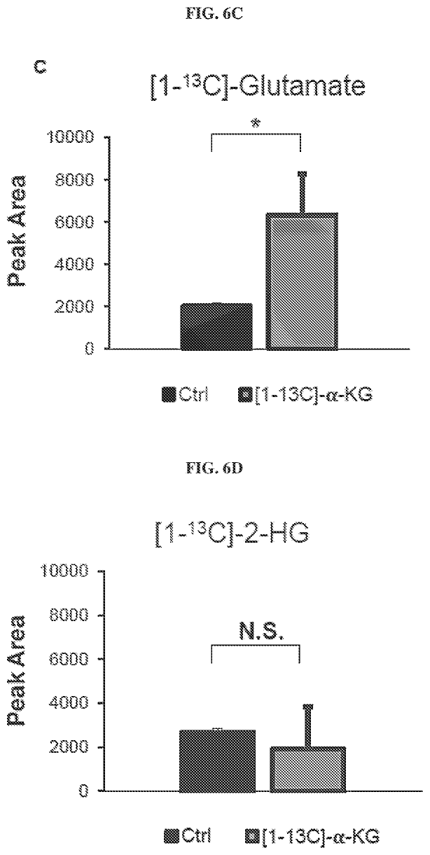

Though [1-¹³C-5-¹′C]-α-KG allowed for the classification of IDH1 status via detection of [1-¹³C]-2-HG in cellulo, the low cell permeability of α-KG led to low concentrations of labeled [1-¹³C]-2-HG. In FIG. 6A the HP-¹³C-MRI of [1-¹³C]-α-KG phantom shows two major peaks at 172 p.p.m. and 181 p.p.m, corresponding to [1-¹³C]-α-KG and [1-¹³C]-α-KG hydrate respectively. The natural abundance C5 and C2 peaks for α-KG were detected as minor peaks at 184 p.p.m. and 208 p.p.m., respectively as shown in FIG. 6A. As shown in FIG. 5, a graph of ¹³C-chemical shift in parts per million (ppm) versus time in seconds illustrating that the use of diethyl ketoglutarate I nHCT 116R132H Cells shows better permeability.

FIG. 6B shows mouse xenografts after a tail injection of hyperpolarized-[1-¹³C]-α-KG into HCT 116 IDH1 R132H, the peaks for α-KG were easily identified, but broad. A peak at 175 p.p.m. corresponding to an unknown contaminant was also present. In FIG. 6B, a small peak at 177 p.p.m. was detected, suggesting that [1-¹³C]-glutamate may be present.

In FIG. 6C, a post-mortem mass spectrometry confirmed that [1-¹³C]-α-KG was metabolized to [1-¹³C]-glutamate in these mouse xenografts, as concentrations of [I-¹³C]-glutamate were significantly increased when comparing xenografts injected with [1 ¹³C]-α-KG to control (N=3 for each group, p=0.019).

FIG. 6D shows that mass spectrometry analysis of the tumors confirmed the insignificant accumulation of labeled [1-¹³C]-α-KG as there was no difference in 11-¹³C]-2-HG concentration in IDH 1R 132H xenografts with, or without, the addition of ¹³C labeled α-KG.

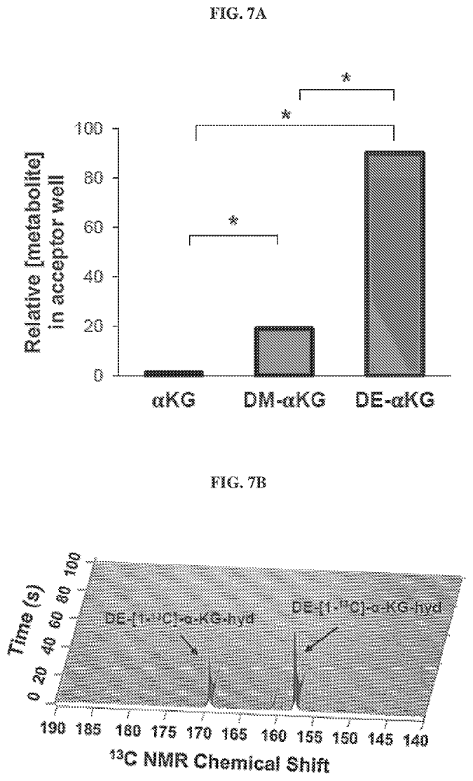

As α-KG is not actively transported across the cell membrane, the negligible degree of product (2-HG) formation is likely a consequence of its reduced intracellular concentration due to its low membrane permeability. A parallel artificial membrane permeability assay (PAMPA) was used to measure the ability of a probe to cross an artificial membrane barrier. FIG. 7A shows that diethyl-[1-¹³C-¹²C]-α-KG (DE-[1-¹³C]α-KG or DE-α-KG) has a 90-fold increase in permeability over α-KG. DE-[1-¹³C]-α-KG also showed an almost 5-fold increase in permeability over dimethyl-α-KG (DM-α-KG). DE-[1-¹³C]-α-KG also forms ethanol after ester cleavage, which is less harmful than the methanol side product formed by cleavage of DM-α-KG.

DE-[1-¹³C]-α-KG is successfully hyperpolarized and is fully polarized within two hours, whereas [1-¹³C-5-¹²C]-αKG required a minimum of five hours to polarize fully (data not shown). The in vitro T1 value of DE-[1-¹³C]-α-KG measured at 3 Tesla was 38.8±0.4 seconds. FIG. 7B shows the resulting HP-¹³C-MRI that shows two major peaks corresponding to DE-[1-¹³C]-α-KG (163 p.p.m.) and DE-[1-¹³C]-α-KG hydrate (174 p.p.m.).

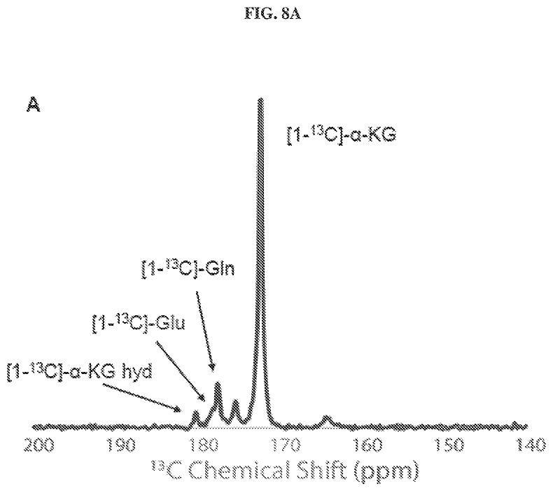

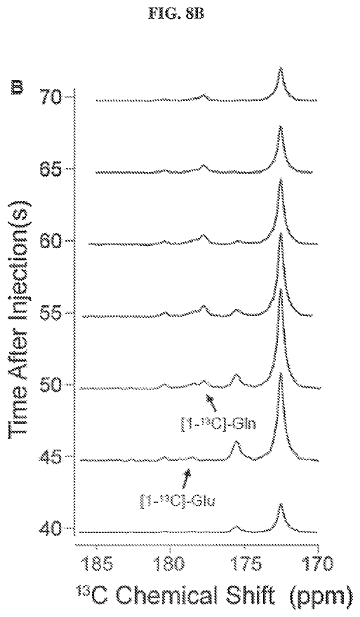

FIG. 8A shows that with DE-[1-¹³C]-α-KG in IDH1 mutant xenografts, the esters were rapidly cleaved to generate [1-¹³C]-α-KG, with the largest peak corresponding to [1-¹³C]-α-KG (172 p.p.m.) and only small peaks remaining for DE-[1-¹³_{C]-α-KG(}174 p.p.m. and 163 p.p.m. for DE-[1-¹³C]-α-KG hydrate and DE-[1-¹³C]-α-KG respectively). FIG. 8A also illustrates the increased permeability of DE-[1-¹³C]-α-KG as the downstream metabolites [1-¹³C]-Glutamate (178 p.p.m.) and [1-¹³C]-Glutamine (177 p.p.m.) were seen in the resulting HP-¹³C-MRI.

FIG. 8B shows that time-course tracing of these peaks showed differential signal decay of the glutamate and glutamine, suggesting that they are not contaminants of the hyperpolarized reagent. With the improved permeability of DE-[1-¹³C]-α-KG, the [1-¹³C]-glutamate and [1-¹³C]-glutamine peaks were able to be resolved. The production of [1-¹³C]-glutamate was seen as a peak at 178 p.p.m. appears 43 seconds after injection of DE-[1-¹³C]-α-KG. The peak at 178 p.p.m. initially increases, but then steadily decreases while a peak at 177 p.p.m, corresponding to [1-¹³C]-glutamine, develops (FIG. 8B).

While this disclosure has been described in connection with what is presently considered to be practical example embodiments, it is to be understood that the disclosure is not limited to the disclosed exemplary embodiments, but, on the contrary, is intended to cover various modifications and equivalent arrangements included within the spirit and scope of the appended claims.

1. A compound of the Formula I 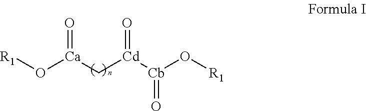
or a pharmaceutically acceptable salt thereof, wherein in Formula I
R₁ is each independently selected from hydrogen, C₁-C₆alkyl, C₃-C₇cycloalkyl, (C₃-C₇cycloalkyl)C₀-C₂alkyl, (heterocycloalkyl)C₀-C₂alkyl, (heteroaryl)C₀-C₂alkyl, or (aryl)C₀-C₂alkyl;
at least two of Ca, Cb, and Cd are each independently chosen from ¹²C and ¹³C; and
n is an integer from 1 to 4.

2. The compound or a pharmaceutically acceptable salt of claim 1, wherein
R₁ is each independently selected from hydrogen or C₁-C₆alkyl;
at least two of Ca, Cb, and Cd are each independently chosen from ¹²C and ¹³C; and
n is 2.

3. The compound or a pharmaceutically acceptable salt of claim 1, where the compound is 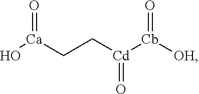
wherein at least two of Ca, Cb, and Cd are each independently chosen from ¹²C and ¹³C,
¹²C is of about 90% to about 99.9% isotope abundance, and
¹³C is of about 80% to about 99.9% isotope abundance.

4. The compound or a pharmaceutically acceptable salt of claim 1, where the compound is one of the following compounds:
diethyl-1-¹³C-5-¹²C-alpha ketoglutarate;
1-¹³C-5-¹²C-alpha ketoglutarate;
diethyl-1-¹³C-5-¹²C-2-hydroxyglutarate;
1-¹³C-5-¹²C-2-hydroxyglutarate;
diethyl-1-¹³C-5-¹²C-glutamate;
1-¹³C-5-¹²C-glutamic acid;
1-¹²C-5-¹³C-alpha ketoglutarate;
diethyl-1-¹²C-5-¹³C-alpha ketoglutarate;
1-¹³C-2-¹²C-5-¹²C-alpha ketoglutarate;
diethyl-1-¹³C-2-¹²C-5-¹²C-alpha ketoglutarate;
1-¹³C-2-¹³C-5-¹²C-alpha ketoglutarate; or
diethyl-1-¹³C-2-¹³C-5-¹²C-alpha ketoglutarate.

5. A pharmaceutical composition comprising an effective amount of an active agent, wherein the active agent is the compound of Formula I, a pharmaceutically acceptable salt, or a combination thereof, and a pharmaceutically acceptable carrier.

6. The pharmaceutical composition of claim 5, wherein said pharmaceutically acceptable carrier is selected from one or more of a sterile liquid or mixture of liquids, including water, saline, aqueous dextrose and related sugar solutions, an alcohol, ethers, an oil, a fatty acid, a fatty acid ester or glyceride, or an acetylated fatty acid glyceride with or without the addition of a pharmaceutically acceptable surfactant, suspending agent, carbomers, methylcellulose, hydroxypropylmethylcellulose, or carboxymethylcellulose, or emulsifying agents and other pharmaceutical adjuvants.

7. The pharmaceutical composition of claim 5, further comprising a pharmaceutically acceptable carrier and a regime of one or more additional chemotherapeutic agents comprising an antineoplastic drug, an antimetabolite, a purine antagonist, a pyrimidine antagonist, taxanes and topoisomerase inhibitors, or biological agents.

8. A method of diagnosing or monitoring a patient suffering from cancer, the method comprising:
administering the pharmaceutical composition comprising an effective amount of an active agent, wherein the active agent is the compound of Formula I, a pharmaceutically acceptable salt, or a combination thereof, together with a pharmaceutically acceptable carrier to the patient; and
diagnosing or monitoring the patient by hyperpolarized ¹³C-MRI.

9. The method of claim 8, wherein the active agent is a hyperpolarized active agent.

10. The method of claim 8, wherein the method further comprises identifying whether the patient has an IDH1 mutation.

11. The method of claim 8, wherein the patient possesses types of cancer known to have IDH1 mutations.

12. The method of claim 8, wherein the method comprises tumor heterogeneity imaging comprising regions of heterogeneity within a single tumor or molecular differences among tumor cell populations at different anatomic locations.

13. The method of claim 8, wherein the method comprises predictive imaging identification of therapies targeting an IDH1 pathway.

14. The method of claim 8, wherein the method comprises monitoring a response of patient's tumor(s) to therapies.

15. The method of claim 8, wherein the method comprises conducting a radiation treatment planning-imaging study with the compound or a pharmaceutically acceptable salt of Formula I fused with a traditional radiation planning computerized tomography (CT) or Magnetic resonance imaging (MRI) scan.

16. The method of claim 8, wherein the method comprises a noninvasive molecular tumor evolution monitoring for molecular changes.

17. The method of claim 8, wherein the method comprises not exposing the patient to an ionizing radiation.

18. The method of claim 8, wherein said pharmaceutical composition further comprises a pharmaceutically acceptable carrier and a regime of one or more other chemotherapeutic agents comprising an antineoplastic drug, an antimetabolite, a purine antagonist, a pyrimidine antagonist, taxanes and topoisomerase inhibitors, or biological agents.

19. The method of claim 8, wherein the patient is human.

20. A process of synthesizing 1-¹³C-5-¹²C-diacid comprising:
treating a ¹³C-morpholine amide, an amine oxide, 3,4,5-trimethyl thiazolium iodide, and a ¹²C-michael acceptor to afford a 1-¹³C-5-¹²C-tricarbonyl compound,
converting the tricarbonyl compound to a 1-¹³C-5-¹²C-diester, and
hydrolyzing the corresponding diester to a corresponding 1-¹³C-5-¹²C-diacid.

21. The process of claim 20, wherein
the ¹³C-morpholine amide is 4—(bromo¹³C-acetyl)morpholine,
the ¹²C-michael acceptor is ¹²C-benzyl acrylate, and
the amine oxide is 4-dimethylaminopyridine N-oxide.

22. The process of claim 20, wherein a 1-¹³C-5-¹²C-diester is di-C₁-C₆alkylester.

23. The process of claim 20, wherein an acid is used to convert a 1-¹³C-5-¹²C-diester to a 1-¹³C-5-¹²C-diacid.

24. The process of claim 20, wherein the acid used is hydrochloric acid.

25. The process of claim 20, wherein 1-¹³C-5-¹²C-diacid is in a keto form, a hydrate form, or a lactone form.

26. The process of claim 20, wherein 1-¹³C-5-¹²C-diacid is 2-oxopentanedioic acid.
